(19) United States
(12) Patent Application Publication
(10) Pub. No.: US 2023/0372573 A1
(43) Pub. Date: Nov. 23, 2023

(54) HEMP-BASED ABSORBENT ARTICLE

(71) Applicant: Better Made Hemp, LLC, Northville, MI (US)

(72) Inventors: Alexandra Blasius, Northville, MI (US); David M. Jackson, Alpharetta, GA (US)

(73) Assignee: Better Made Hemp, LLC, Northville, MI (US)

(21) Appl. No.: 18/318,153

(22) Filed: May 16, 2023

Related U.S. Application Data
(60) Provisional application No. 63/342,709, filed on May 17, 2022.

(51) Int. Cl. A61L 15/40; A61F 13/20; A61L 15/62

(52) U.S. Cl. CPC A61L 15/40; A61F 13/202; A61F 13/2085; A61L 15/62

(57) ABSTRACT

Hemp-based absorbent articles are disclosed. The absorbent article can be a tampon or pad for feminine hygiene. The absorbent article can be flushable or compostable. In one example, the absorbent article is a flushable tampon for feminine hygiene having a nonwoven material comprising hemp with a hemp fiber length in a range of about 8 mm to about 15 mm. The nonwoven material can include an absorbent ribbon, a string, and an overwrap.

### CROSS-REFERENCE TO RELATED APPLICATION

This application is a U.S. Non-provisional patent application, being filed on May 16, 2023, claiming priority and benefit of provisional patent application 63/342,709 filed on May 17, 2022, the contents of which are incorporated herein in their entirety.

### TECHNICAL FIELD

This application generally relates to absorbent articles for feminine hygiene and, in particular, to hemp-based absorbent articles.

### BACKGROUND

Absorbent articles are used to absorb and hold bodily exudates (e.g., urine, menses) and/or fluids. There are many different types of absorbent articles, which may include diapers, pants, sanitary napkins, and tampons, for example. Some absorbent articles are inserted inside a body of a wearer, particularly tampons, to prevent leakage of bodily exudates. During a menstrual cycle, a wearer may insert a tampon and leave the tampon inside the body for hours at a time to absorb and hold the bodily exudates. It is often desirable to prevent any leakage during use and, at the same time, prevent any bacterial buildup that could potentially harm the wearer.

During the menstrual cycle, blood is a major reason for germs to grow which causes unpleasant odor and raises the possibility of bacterial buildup. Some absorbent articles include multiple layers that are capable of quickly absorbing fluids and subsequently passing the fluids to the next layer to retain the fluids. With multiple layers, the blood is spread out between the layers such that germs are less likely to grow and build up. By having an absorbent article positioned inside the body for a certain amount of time, it is desirable that the absorbent article is made of material and layers that do not compromise the health of the wearer.

Most women experience painful symptoms or discomfort leading up to, during, and after menstruation. For some, symptoms are so severe that it becomes difficult to carry out normal tasks of daily life. Symptoms may include muscle aches, headaches, joint pain, bloating, acne, abdominal cramps, constipation, tenderness, lower back pain, and fatigue. Conventional absorbent articles may lack efficient composition to alleviate any of the aforementioned symptoms.

Leakage may occur due to the less than optimal fit of the absorbent article. Because of the layers' many properties, such as absorbency, flexibility, and structure, it may be desirable to incorporate a more efficient structure into such articles to prevent any bacterial buildup and, at the same time, prevent any leakage.

Finally, absorbent articles are a single use product. Once the absorbent article is used, the wearer throws the absorbent article away. In such instances, the absorbent article may not be easily degradable or environmentally friendly. In other instances, the absorbent article may not be made from renewable resources. Therefore, an absorbent article providing features designed to overcome one or more of the aforementioned challenges is desired.

### SUMMARY

Embodiments for hemp-based absorbent articles are disclosed. The absorbent article can be a tampon or pad for feminine hygiene. The absorbent article can be flushable or compostable. In one example, the absorbent article is a flushable tampon for feminine hygiene. The tampon can include a nonwoven material comprising hemp with a hemp fiber length in a range of about 8 mm to about 15 mm. The nonwoven material can include an absorbent ribbon, a string, and an overwrap for the tampon.

In other embodiments, the absorbent article is a compostable tampon. The tampon can comprise a nonwoven material that is carded and comprises hemp with a hemp fiber length in a range of about 40 mm to about 55 mm, in some versions. The nonwoven material can be formed by hydroentangling bonding, and comprises an absorbent ribbon, a string, and an overwrap.

In other embodiments, the absorbent article is a pad for feminine hygiene. The pad comprising a nonwoven material comprising hemp, having a hemp fiber length in a range of about 8 mm to about 15 mm.

The terminology used herein is for the purpose of describing particular example embodiments only and is not intended to be limiting. As used herein, the singular forms “a,” “an,” and “the” may be intended to include the plural forms as well, unless the context clearly indicates otherwise. The terms “comprises,” “comprising,” “including,” and “having,” are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring their performance in the particular order discussed or illustrated, unless specifically identified as an order of performance. It is also understood that additional or alternative steps may be employed.

It can be advantageous to set forth definitions of certain words and phrases used throughout this patent document. The term “communicate,” as well as derivatives thereof, encompasses both direct and indirect communication. The terms “include” and “comprise,” as well as derivatives thereof, mean inclusion without limitation. The term “or” is inclusive, meaning and/or. The phrase “associated with,” as well as derivatives thereof, can mean to include, be included within, interconnect with, contain, be contained within, connect to or with, couple to or with, be communicable with, cooperate with, interleave, juxtapose, be proximate to, be bound to or with, have, have a property of, have a relationship to or with, or the like. The phrase “at least one of,” when used with a list of items, means that different combinations of one or more of the listed items can be used, and only one item in the list can be needed. For example, “at least one of: A, B, and C” includes any of the following combinations: A, B, C, A and B, A and C, B and C, and A and B and C.

When an element or layer is referred to as being “on,” “engaged to,” “connected to,” or “coupled to” another element or layer, it may be directly on, engaged, connected or coupled to the other element or layer, or intervening elements or layers may be present. In contrast, when an element is referred to as being “directly on,” “directly engaged to,” “directly connected to,” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the example embodiments.

Spatially relative terms, such as “inner,” “outer,” “beneath,” “below,” “lower,” “above,” “upper,” “top”, “bottom,” and the like, may be used herein for ease of description to describe one element's or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated degrees or at other orientations) and the spatially relative descriptions used herein interpreted accordingly.

### BRIEF DESCRIPTION OF THE DRAWINGS

FIG. 1 is a perspective view of an embodiment of an absorbent article, such as a tampon for feminine hygiene.

FIG. 2 is a perspective view of alternate embodiment of an absorbent article, such as a pad for feminine hygiene.

FIG. 3 is a perspective view of alternate embodiment of an absorbent article, such as a pad for feminine hygiene.

### DETAILED DESCRIPTION

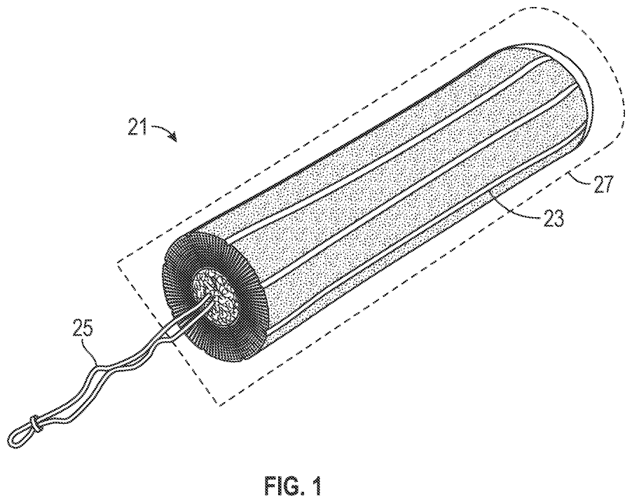

FIGS. 1-3 depict various embodiments of absorbent articles disclosed herein. For example, an absorbent article to prevent unwanted leakage and bacterial backup is provided. A wearer may utilize an absorbent article during menstruation. In one embodiment, an absorbent article 21 is a tampon.

Tampon

Embodiments of the absorbent article 21, shown in FIG. 1, disclose a flushable tampon for feminine hygiene. The tampon can comprise a nonwoven material comprising hemp with a hemp fiber length in a range of about 8 mm to about 15 mm, in some embodiments. The hemp fiber length is not greater than 12 mm, in other embodiments. The short length of the fiber is essential in creating a flushable absorbent article, since longer length fiber would not be able to disperse in water. In addition, the nonwoven material can comprise an absorbent ribbon 23 and a string 25. The absorbent ribbon 23 can be at least one of carded hemp, bleached cotton, viscose rayon, or a combination thereof. In one embodiment, the absorbent ribbon 23 comprises about 25% hemp and about 75% viscose rayon, with respect to the combination of total absorbent fibers used. In another embodiment, the absorbent ribbon 23 comprises about 50% hemp and about 50% viscose rayon, with respect to the combination of total absorbent fibers used.

An example of the carding process is performed by an Andritz nonwoven machinery, of Montbonnot St. Martin, France. Another example of the carding process is performed by a Bonino Carding Machine, of Sandigliano (Biella), Italy. In one example, U.S. Pat. No. 4,041,948 (which is incorporated herein by reference in its entirety) discloses winding the absorbent ribbon (absorbent) and cover in to a cullet shape. Other examples include U.S. Pat. Nos. 5,948,710 and 8,530,721, each of which is incorporated herein by reference in its entirety.

In these versions, the nonwoven can be made typically on a very wide machine and slit into strips to be made into the tampon. Whether it is a bullet or a lotus flower shaped tampon can depend on the sizes needed. The absorbent ribbon and overwrap can be made in the same way. A absorbent ribbon can be carded at a very light basis weight and cross-machine gathered into the ribbon width needed. The Truetzschler and Andritz companies makes typical card lines. A absorbent ribbon can be made from carded fibers in a unique process, such as with the Bonino company. The carded material can be gathered to make the tampon ribbon.

The absorbent ribbon 23 can comprises a sliver ribbon having a width in a range of about 45 mm to about 55 mm, such as about 51 mm, in some versions. The string 25 can comprise at least one of polyester or cotton material.

An overwrap 27 for the tampon also can be included. The overwrap 27 can comprise a carded or spunlaced nonwoven fabric. The overwrap 27 can comprise fibers having a basis weight in a range of about 15 g/m² to about 25 g/m², such as about 20 g/m². In another example, the overwrap 27 can include fibers having a basis weight that is not greater than about 40 g/m². In some embodiments, the overwrap fibers can comprise a length in a range of about 42 mm to about 52 mm.

Examples of the nonwoven material can be produced by an airlaid or air forming process. An example of the air forming process is performed by a Campen Machine of Hornslet, Denmark. The nonwoven material can be hydrogen bonded. In some embodiments, the nonwoven material can comprise a water dispersible bi-component thermal binder fiber. For example, this can include a low content of the bi-component, e.g., polypropylene and polyethylene, such as Fibervisions ESC type. The nonwoven material can comprise no greater than about 5% by weight of the bi-component thermal binder fiber, in one example. In another example, the nonwoven material can comprise a range of about 2% to about 5% by weight of the bi-component thermal binder fiber.

Embodiments of the tampon can include a cover material, or not include a cover material. In an example, the cover material can comprise the nonwoven material. The cover material can comprise a basis weight of not greater than about 40 g/m², in one version. In other versions, the cover material can comprise a basis weight in a range of about 15 g/m² to about 25 g/m².

Other embodiments of the nonwoven material can comprise an absorbent ribbon 23 having a width of about 50 mm. An example of the absorbent ribbon process is performed by a Bonino Carding Machine, of Sandigliano (Biella), Italy. The absorbent ribbon 23, having a width of about 50 mm, comprises a basis weight in a range of about 8 grams per meter of length to about 11 grams per meter of length, in some examples. These values are made with respect to grams per running length of the formed absorbent articles. In another example, the absorbent ribbon can comprise a basis weight of about 9.5 grams per meter of length.

Other embodiments of the absorbent article 21 can be compostable. Being compostable does not necessarily mean that the product is flushable, but rather that the article is capable of naturally degrading, with or without the assistance of naturally occurring microorganisms. For example, the absorbent article 21 can be a tampon for feminine hygiene. The tampon can comprise a nonwoven material that is carded and comprises hemp with a hemp fiber length in a range of about 40 mm to about 55 mm, in some versions. The nonwoven material can be formed by hydroentangling bonding, and comprises an absorbent ribbon 23 and a string 25. An example of the hydroentangling process, sometimes also referred to as spunlacing, is performed by an Andritz nonwoven machinery, of Montbonnot St. Martin, France. An overwrap 27 for the tampon also can be included.

In one example, the nonwoven material can comprise up to 100% hemp fiber, or up to 90%, or up to 80% or up to 70%, or up to 60%, or up to 50%, or up to 40%, or up to 30%, or up to 20% or up to 10%, or an amount greater than 10%, or greater than 20%, or greater than 30%, or greater than 30%, or greater than 40%, or greater than 50%, or greater than 60% or greater than 70%, or greater than 80%, or greater than 90% of hemp fiber, or any value between said ranges.

Versions of the nonwoven material can comprise a blend of materials comprising at least one of viscose rayon, cotton or polylactic acid (PLA) fibers. The nonwoven material can be produced by an air forming process. In addition or alternatively, the nonwoven material can be hydrogen bonded. The nonwoven material comprise a water soluble or dispersible bi-component thermal binder fiber, as described above. In one example, the nonwoven material can comprise no greater than about 5% by weight of the bi-component thermal binder fiber. In another example, the nonwoven material can comprise a range of about 2% to about 5% by weight of the bi-component thermal binder fiber. In another embodiment, water dispersible polyvinyl alcohol fibers are incorporated as the binder component for the nonwoven material. In one embodiment, the polyvinyl binder fibers have a fiber length in the range of 8 mm to 15 mm. The nonwoven material can comprise the polyvinyl alcohol fibers in about 5% to about 20% by weight, of the total weight of the nonwoven material, with the remainder being comprised of a combination of absorbent fibers, as discussed above, including hemp or combination of hemp and viscose rayon, in some embodiments.

The tampon may or may not comprise a cover material. In an example, the cover material can comprise the nonwoven material. The cover material can comprise a basis weight of not greater than about 40 g/m², or a basis weight in a range of about 15 g/m² to about 25 g/m², in some versions.

In some embodiments of the absorbent article 21, the nonwoven material can comprise an absorbent ribbon absorbent ribbon having a width of about 50 mm. The absorbent ribbon absorbent ribbon 23 can comprise a basis weight in a range of about 8 grams per meter of length to about 11 grams per meter of length, such as about 9.5 grams per meter of length, in some examples. The overwrap 27 can comprise a carded or spunlaced nonwoven material. Embodiments of the overwrap 27 can include fibers having a basis weight in a range of about 15 g/m² to about 25 g/m², such as about 20 g/m². The overwrap 27 can comprise fibers having a basis weight that is not greater than about 40 g/m². In some embodiments, the overwrap fibers can have a length in a range of about 42 mm to about 52 mm.

The absorbent ribbon 23 can comprise at least one of carded hemp, bleached cotton or viscose rayon, or combination thereof. For example, the absorbent ribbon 23 can comprise about 25% hemp and about 75% viscose rayon. In another embodiment, the absorbent ribbon 23 comprises about 50% hemp and about 50% viscose rayon. The absorbent ribbon 23 can comprise a sliver ribbon having a width in a range of about 45 mm to about 55 mm, such as about 51 mm, in some examples. The string 25 can comprise at least one of polyester or cotton.

Pad

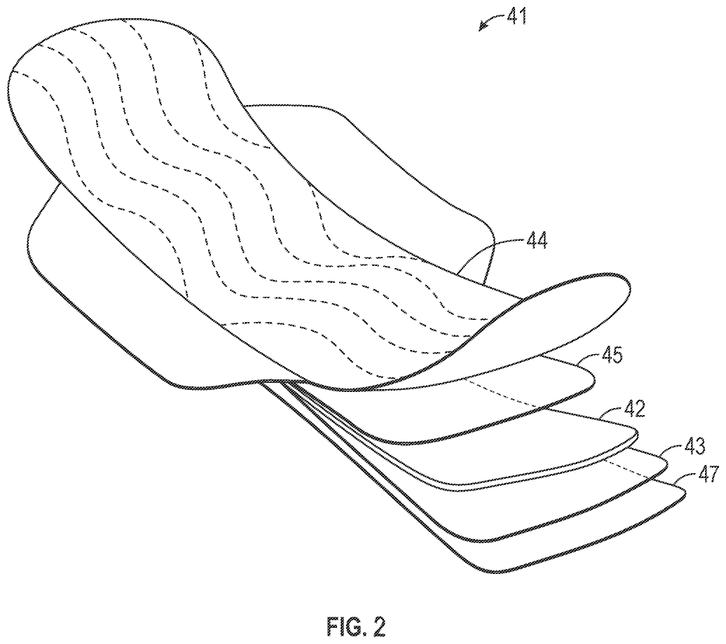

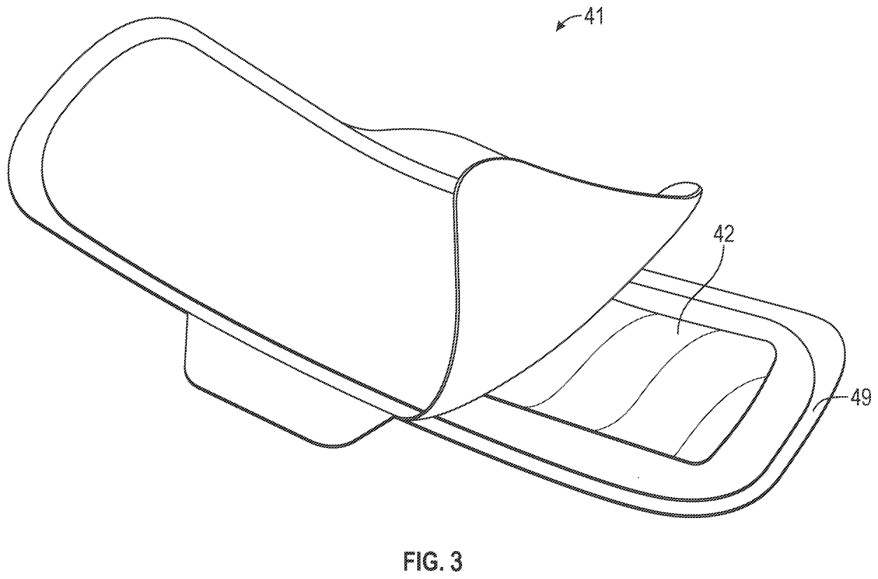

Referring now to FIGS. 2-3, still other embodiments of an absorbent article 41 can be flushable. For example, the absorbent article 41 can comprise a pad for feminine hygiene. Embodiments of the pad can comprise a nonwoven material 42 comprising hemp and having a hemp fiber length in a range of about 8 mm to about 15 mm. The absorbent article of claim 1, wherein the hemp fiber length is not greater than 12 mm. The nonwoven material 42 can be produced by an air forming process. The nonwoven material 42 can be hydrogen bonded.

Embodiments of the nonwoven material 42 can comprise a bi-component thermal binder fiber, such as those described above. For example, the nonwoven material 42 can comprise no greater than about 5% by weight of the bi-component thermal binder fiber, and the bi-component thermal binder fiber can include polypropylene and polyethylene. The nonwoven material 42 can comprise a range of about 2% to about 5% by weight of the low contact, bi-component thermal binder fiber, in some versions. In other versions, the nonwoven material 42 can comprise an absorbent basis weight in a range of about 125 g/m² to about 250 g/m².

In other embodiments, the pad can comprise a barrier film 43 comprising polyvinyl alcohol (PVOH). The nonwoven material 42 can comprise greater than about 50% hemp fiber, such as up to 100% hemp fiber. The nonwoven material 42 can comprise a blend of materials comprising at least one of viscose rayon, cotton or polylactic acid (PLA) fibers.

Embodiments of the absorbent article 41 can be compostable. The absorbent article 41 can be a pad for feminine hygiene that comprises a nonwoven material 42 that is carded and comprises hemp and a hemp fiber length is in a range of about 40 mm to about 55 mm. In one example, the nonwoven material 42 is formed by hydroentangling bonding. Versions of the pad can include a barrier film 43 comprising polylactic acid (PLA). The nonwoven material 42 can comprise any of the other features, ranges and components described herein for other embodiments of the pad.

Embodiments of the pad can further include one or more of the following items: a cover 44 (e.g., nonwoven or perforated film), a transfer layer 45, a release paper 47 or a underlying adhesive layer 49.

The absorbent article can include a body portion with a proximal end and a distal end with a string extending from the distal end of the body portion. The string is configured to aid the wearer in removal of the absorbent article from the wearer's body. The proximal end is adapted to be the leading end during insertion into the wearer's body and enters the wearer's body first. The distal end is adapted to be the tailing end during insertion and enters the wearer's body last. After usage, the wearer, using one hand, may pull on the string extending from the distal end to remove the absorbent article with the proximal end exiting the wearer's body last.

The body portion may be a core that may be substantially enclosed by a plurality of layers. More specifically, the plurality of layers can each comprise one or more absorbent petals having petal ends. The plurality of layers is configured to aid in leakage prevention during use or when exposed to bodily fluids. The plurality of layers and/or one or more absorbent petals prevent substantial leakage during usage or when exposed to bodily fluids by absorbing fluids from the layer of the preceding one of the plurality of layers. It will be appreciated that the term “fluids” may be interchangeable with “exudates,” unless otherwise stated.

The core may be made in various shapes and sizes. For example, the core can extend along an axis and has a generally cylindrical shape. In another example, the core may be cone shaped with the distal end being wider than the proximal end. It should be understood that the core may extend the entire length of the absorbent article.

The core may change shape and/or size during usage. For example, once inserted, the core may expand from its cylindrical shape and conform to contours of the wearer's body. As used herein, the term “core” refers to a material or combination of materials suitable for absorbing, distributing, collecting, and storing any type of bodily exudates. The shape and size of the core may be altered or convertible to meet any absorbent capacity requirements. Other shapes and sizes of the core are contemplated.

The petal ends may comprise any shape to facilitate migration of bodily fluids in a manner that enhances the cleanliness and/or health of the wearer by absorbing and collecting bodily fluids without allowing the fluids to flow back to the wearer.

One or more absorbent petals and/or petal ends, when introduced to a fluid, absorbs and transitions from the compacted configuration to the expanded configuration. It will be understood that any portions of the plurality of layers may absorb and/or transition. There can be a transition between the compacted configuration to the expanded configuration. In various configurations, the absorbent article resembles a flower in the expanded configuration. In such configurations, the absorbent article “blooms” or “flowers” as absorption of fluid occurs. Of course, alternative configurations of the absorbent article 10 may expand into a variety of different shapes.

In the expanded configuration, the petal ends enable migration of the bodily fluid in a controlled manner by directly streaming the fluids down the petals. For example, the petal ends may absorb bodily fluid and stream the fluids down the petal ends towards the core to prevent fluid leakage by a layer of the preceding one of the plurality of layers. The combination of the core and the plurality of layers collects and/or absorbs the bodily exudates and thereby prevents fluid leakage to assist in leakage protection. Other shapes and methods of migration of bodily fluids via the petal ends are contemplated.

One or more absorbent petals and/or petal ends are movable to conform to the contours of the wearer's body to control the flow of bodily fluid and prevent leakage.

The absorbent article may comprise any type of natural material, including natural fibers. Use of natural materials is advantageous as natural materials are a sustainable resource, as they are renewable, biodegradable, and carbon neutral. Further, natural materials of the absorbent article may be used without depleting or damaging the environment. In some configurations, the absorbent article, a packaging assembly, and/or an applicator may consist of, consist essentially of, or comprise biodegradable material. The absorbent article, the packaging assembly (e.g. wrapper) and/or an applicator may be biodegradable, compostable, or flushable. The applicator may be made from a combination of thermoplastic materials, especially a blend of water-dispersible polymers and biodegradable polymers to result in disintegrable applicators. Such applicators are easily disposed with minimal or no environmental harm.

In yet another example, the applicator may comprise, consist essentially of, or consist of, a renewable polymer formed from renewable resources (e.g. a polymer formed from plant-based and not petroleum-based based monomers). It will be appreciated that the applicator may comprise any amount of biodegradable polymer, water-dispersible polymers, and/or renewable polymer.

It is contemplated that the absorbent article, the packaging assembly of the absorbent article, and/or the applicator may be constructed in any blend, composite, shape, or configuration using water-dispersible and/or biodegradable polymers, and any other desired or optional ingredient to provide benefits to the absorbent article or to the materials used in making the absorbent article. Such benefits include, but are not limited to, stability, flexibility, resiliency, toughness, workability, odor control, improved strength, improved modulus, improved melt flow characteristics, and/or dispensability of the thermoplastic compositions. The optional ingredient may include, but not limited to, plasticizing agents, antioxidants, slip agents, flow promoters, processing aids, pigments or colorants, mold release agents, coating agents, gelling agents, antistatic agents, dispersing agents, lubricants, surfactants, odor masking agents, opacifying agents such as aluminum oxide, dyes, viscosity modifiers, waxes, elastomers, and mixtures thereof.

It will further be appreciated that the term “biodegradable” as used herein refers to materials that when disposed of after use will physically and/or biologically decompose using known degradation procedures including, but not limited to, aerobic, anaerobic, and microbial digestion processes. The biodegradable materials described herein include those degradable water-insoluble materials that will also physically and/or biologically decompose after disposal.

In one configuration, a suitable natural material is hemp-based. Hemp is derived from the Cannabis sativa plant species. The Cannabis sativa plant is a multi-purpose plant. For example, the plant may be domesticated for fiber in the stem, processed for oil from the seeds, and grown for other purposes including, but not limited to, medicinal and therapeutic purposes. Specifically, hemp can be grown as a fiber and/or seed. Hemp stalk and seed(s) are harvested to produce products including, but not limited to, textiles, paper, building materials, foods, personal hygiene products and seed cakes.

In various configurations, the absorbent article may include hemp-based material in an amount of up to 50, 60, 70, 80, 90, or 100, weight percent based on the total weight of the absorbent article. In various configurations, the hemp-based material is present in an amount of 1 to 20, 21 to 40, 41 to 60, 61 to 80, 81 to 99, weight percent based on a total weight of the absorbent article 10. In additional non-limiting configurations, all values and ranges of values described herein are hereby expressly contemplated.

It will be appreciated that, the absorbent article may comprise one or more hemp-based materials selected from fiber, oil, extract, probiotic, remedy, essence and combinations thereof. It will further be appreciated that the hemp-based material may be a strain selected from Cannabis indica strain, Cannabis sativa, hybrid strain, high-CBD strain, and high-THC strain.

The Cannabis sativa plant may produce hemp fibers, hemp oil, and other plant materials. Hemp fibers may be derived from the stalk and hemp oil may be produced from the seed. It will be appreciated that hemp fiber, hemp oil, and any other hemp material may be processed, produced, or the like in any suitable method. It will be understood that other plant material derived from the Cannabis sativa plant are contemplated for use in the absorbent article.

Any component, part, and/or feature of the absorbent article may comprise hemp. For example, the plurality of layers may consist of, consist essentially of, or comprise hemp fiber. The hemp fiber and/or thread may be knitted, twisted, weaved and the like to make products for use by a wearer. It will be understood that products include, but are not limited to, absorbent articles and clothing.

In another example, the core may consist of, consist essentially of, or comprise hemp fiber and/or hemp oil. The core may be formed of hemp fiber and infused with hemp oil for therapeutic or medicinal purposes. In yet another example, the string may consist of, consist essentially of, or comprise hemp fiber. In this configuration, the string may comprise one or more strands of hemp fiber. The string may be crocheted, cabled, twisted, or braided or any combination thereof. The individual strands of hemp fiber can remain in substantially continuous contact with one another along the entire length of the string. It will be appreciated that the string may comprise any size, shape, length and width.

In yet another example, the absorbent article, including the plurality of layers, the core, and the string may consist of, consist essentially of, or comprise hemp fiber. Significant advantages, including the above-mentioned advantages, are contemplated by providing the absorbent article comprising hemp. It is contemplated that the absorbent article including any components or features described above may comprise any other natural fiber. Other natural fibers including, but not limited to, cotton, bamboo, silk, sisal, wool, ramie, flax, coir are contemplated. While various combinations of the plurality of layers, core and string comprising hemp fiber are discussed, they are not intended to be limiting. The combinations described above are merely exemplary configurations of the absorbent article.

As mentioned above, the Cannabis sativa plant may produce hemp oil for technical hemp products. More specifically, the seed(s) may be crushed and processed to produce hemp oil. It will be appreciated that other types of technical hemp products including, but not limited to, oil paints, printing inks, solvents, lubricants, coatings, or any combinations thereof may be applied to the absorbent article.

From an environmental perspective, coating systems including excessive amounts of volatile fluids may be harmful to the environment. Additionally, a coating system may be formed from non-renewable resources. The absorbent article may include a coating that is eco-friendly and/or formed from renewable resources. A suitable coating may be hemp-based. In some configurations, the absorbent article may comprise a hemp coating. In one example, the plurality of layers may comprise a coating produced from the hemp seeds, hemp leaves, or other material from the Cannabis sativa plant. The coating may be applied to the plurality of layers in any manner. Further, the hemp coating may be applied to enhance the aesthetic appeal by accentuating the plurality of layers. Other applications of the hemp coating are contemplated. It will be appreciated that the hemp-based coating may be applied to any portion of the absorbent article.

Alternatively, or additionally, the hemp coating may be applied for therapeutic applications. For example, the coating may be applied to the core to help remedy menstrual cramping as the core is inserted into the wearer's body. In another example, the coating may be applied to the plurality of layers which may be in contact with the wearer's skin. The hemp-based coating may be applied to the absorbent article using any suitable method. Other components or features of the absorbent article including, but limited to, the protective strip and a wrapper may also comprise hemp-based material.

From a therapeutic perspective, natural oils may be extracted from a plant and applied to absorbent articles to alleviate or relieve pains, aches, cramps related to menstruation. Natural oil is an oil obtained from a renewable source. Some natural oils include, but are not limited to, canola oil, castor oil, coconut oil, cottonseed oil, olive oil, palm oil, peanut oil, rapeseed oil, soybean oil, hemp oil and combinations thereof. One particularly suitable natural oil is hemp oil, which is derived from the plant Cannabis sativa, as mentioned above.

In some configurations, the absorbent article may be infused with hemp oil produced or extracted from the Cannabis sativa plant. The plurality of layers and/or the core may be infused with hemp oil for therapeutic remedies including, but not limited to, healing, moisturizing, or relieving the wearer of any menstrual pain or cramping. Further, the hemp oil may be infused to result in less odor or fouling. Other applications of the hemp oil are contemplated.

In a preferred configuration, the absorbent article may be infused with cannabidiol (CBD) extracted from a Cannabis plant. For example, CBD oil may be extracted. The CBD oil applied to the absorbent article may be a naturally occurring and/or synthetically prepared compound. The amount of CBD present in a dosage form may vary over a wide range, but by way of example often ranges from about 1 to about 300 mg, more usually from about 2 to about 250 mg, and typically from about 3 to about 200 mg, about 4 to about 180 mg, or about 5 to about 160 mg.

An advantage of using CBD is the non-psychoactive effects and rather helps regulate body functions such as sleep, immune-system responses, and pain. For example, the CBD oil may be applied under conditions sufficient to infuse the CBD oil into the layers, the string and/or the core for therapeutic remedies including, but not limited to, healing and relieving the wearer of any menstrual pain or cramping. In another example, the CBD oil may be applied to a surface of each layer. In yet another example, the CBD oil may be applied under conditions sufficient to infuse the CBD oil between the layers, between each strand of the string and/or between layers of the core. It will be appreciated that the CBD oil may be infused and/or applied to the absorbent article in any suitable manner.

If included, the hemp-based coating and/or oil may be included in an amount of up to 1, 2, 3, 4, 5, 6, 7, 8, 9, 10, 20, 30, 40, or 50, weight percent based on the total weight of the absorbent article. Likewise, hemp-based coating and/or oil may be included in an amount of from 0.1 to 70, 0.5 to 10, 1 to 5, or 1 to 10, weight percent based on the total weight of the absorbent article. The amount of the hemp-based coating and/or oil may vary outside of the ranges described above. In additional non-limited embodiments, all values and ranges of values within are hereby expressly contemplated.

The absorbent article may include other aesthetic and therapeutic properties to result in less fouling, smell, pain, abrasion or harm of the wearer. It is also contemplated that an essential oil may be infused with the layers and/or the core for therapeutic remedies. Other types of oil and/or coating may be infused with or applied to the layers, the core and/or any portions of the absorbent article.

The absorbent article may comprise any type of lotion composition. In a preferred configuration, the absorbent article may comprise a hemp-based lotion composition. The lotion may be applied in any type of manner, pattern, and/or amount. For example, the lotion may be applied with small droplets, discrete dots, stripes that run along the extension of the layers, the core and/or any portions of the absorbent article. It will be appreciated that any oil, lotion or the like may be applied to the absorbent article in different amounts, locations, patterns of distribution, and/or methods. It will further be appreciated that any oil, lotion, or the like may be applied to the absorbent article for therapeutic remedies including, but not limited to, healing, moisturizing, or relieving the wearer.

After usage of the absorbent article, the wearer may utilize the string to remove the absorbent article from inside the wearer's body. The string is attached to the distal end of the absorbent article and configured to be grippable for removal from the wearer's body. The string may comprise any variation in diameter and cross-section along the length of the absorbent article to provide a more enhanced grippability of the string. For removal, the wearer may grasp the string anywhere along the string's extension and pull the absorbent article out of the wearer's body.

Other embodiments are outlined in the various clauses listed below:
1. An absorbent article that is flushable, comprising:
   a tampon for feminine hygiene, the tampon comprising a nonwoven material comprising hemp with a hemp fiber length in a range of about 8 mm to about 15 mm, and the nonwoven material comprises an absorbent ribbon and a string; and
   an overwrap for the tampon.
2. The absorbent article wherein the nonwoven material is produced by an air forming process.
3. The absorbent article wherein the nonwoven material is hydrogen bonded.
4. The absorbent article wherein the nonwoven material comprises a water soluble bi-component thermal binder fiber.
5. The absorbent article wherein the nonwoven material comprises no greater than about 5% by weight of the water soluble bi-component thermal binder fiber, and the bi-component thermal binder fiber comprises polypropylene and polyethylene.
6. The absorbent article wherein the nonwoven material comprises a range of about 2% to about 5% by weight of the bi-component thermal binder fiber.
7. The absorbent article wherein the tampon does not comprise a cover material.
8. The absorbent article wherein the tampon further comprises a cover material.
9. The absorbent article wherein the cover material comprises the nonwoven material.
10. The absorbent article wherein the cover material comprises a basis weight of not greater than about 40 g/m².
11. The absorbent article wherein the cover material comprises a basis weight in a range of about 15 g/m² to about 25 g/m².
12. The absorbent article wherein the hemp fiber length is not greater than 12 mm.
13. The absorbent article wherein the nonwoven material comprises a absorbent ribbon having a width of about 50 mm.
14. The absorbent article wherein the absorbent ribbon comprises a basis weight in a range of about 8 grams per meter of length to about 11 grams per meter of length.
15. The absorbent article wherein the absorbent ribbon comprises a basis weight of about 9.5 grams per meter of length.
16. The absorbent article wherein the overwrap comprises a carded or spunlaced nonwoven.
17. The absorbent article wherein the overwrap comprises fibers having a basis weight in a range of about 15 g/m² to about 25 g/m².
18. The absorbent article wherein the overwrap comprises fibers having a basis weight that is not greater than about 40 g/m².
19. The absorbent article wherein the overwrap fibers comprise a length in a range of about 42 mm to about 52 mm.
20. The absorbent article wherein the absorbent ribbon comprises carded hemp, bleached cotton and viscose rayon or a combination thereof.
21. The absorbent article wherein the absorbent ribbon comprises about 25% hemp and about 75% viscose rayon.
22. The absorbent article wherein the absorbent ribbon comprises a sliver ribbon having a width in a range of about 45 mm to about 55 mm.
23. The absorbent article wherein the string comprises at least one of polyester or cotton.
24. An absorbent article that is compostable, comprising:
   a tampon for feminine hygiene, the tampon comprising a nonwoven material that is carded and comprises hemp with a hemp fiber length is in a range of about 40 mm to about 55 mm, and the nonwoven material comprises hydroentangling bonding, an absorbent ribbon and a string; and
   an overwrap for the tampon.
25. The absorbent article wherein the nonwoven material comprises up to 100% hemp fiber.
26. The absorbent article wherein the nonwoven material comprises greater than about 50% hemp fiber.
27. The absorbent article wherein the nonwoven material comprises a blend of materials comprising at least one of viscose rayon, cotton or polylactic acid (PLA) fibers.
28. The absorbent article wherein the nonwoven material is produced by an air forming process.
29. The absorbent article wherein the nonwoven material is hydrogen bonded.
30. The absorbent article wherein the nonwoven material comprises a water soluble bi-component thermal binder fiber.
31. The absorbent article wherein the nonwoven material comprises no greater than about 5% by weight of the water soluble bi-component thermal binder fiber, and the bi-component thermal binder fiber comprises polypropylene and polyethylene.
32. The absorbent article wherein the nonwoven material comprises a range of about 2% to about 5% by weight of the bi-component thermal binder fiber.
33. The absorbent article wherein the tampon does not comprise a cover material.
34. The absorbent article wherein the tampon further comprises a cover material.
35. The absorbent article wherein the cover material comprises the nonwoven material.
36. The absorbent article wherein the cover material comprises a basis weight of not greater than about 40 g/m².
37. The absorbent article wherein the cover material comprises a basis weight in a range of about 15 g/m² to about 25 g/m².
38. The absorbent article wherein the nonwoven material comprises an absorbent ribbon having a width of about 50 mm.
39. The absorbent article wherein the absorbent ribbon comprises a basis weight in a range of about 8 grams per meter of length to about 11 grams per meter of length.
40. The absorbent article wherein the overwrap comprises a carded or spunlaced nonwoven.
41. The absorbent article wherein the overwrap comprises fibers having a basis weight in a range of about 15 g/m² to about 25 g/m².
42. The absorbent article wherein the overwrap comprises fibers having a basis weight that is not greater than about 40 g/m².
43. The absorbent article wherein the overwrap fibers comprise a length in a range of about 42 mm to about 52 mm.
44. The absorbent article wherein the absorbent ribbon comprises carded hemp, bleached cotton, viscose rayon, or a combination thereof.
45. The absorbent article wherein the absorbent ribbon comprises about 25% hemp and about 75% viscose rayon.
46. The absorbent article wherein the absorbent ribbon comprises a sliver ribbon having a width in a range of about 45 mm to about 55 mm.
47. The absorbent article wherein the string comprises at least one of polyester or cotton.
48. The absorbent article wherein the tampon comprises a lotus flower shape.
1. An absorbent article that is flushable, comprising:
   a pad for feminine hygiene, the pad comprising a nonwoven material comprising hemp and having a hemp fiber length in a range of about 8 mm to about 15 mm.
2. The absorbent article wherein the hemp fiber length is not greater than 12 mm.
3. The absorbent article wherein the nonwoven material is produced by an air forming process.
4. The absorbent article wherein the nonwoven material is hydrogen bonded.
5. The absorbent article wherein the nonwoven material comprises a bi-component thermal binder fiber.
6. The absorbent article wherein the nonwoven material comprises no greater than about 5% by weight of the bi-component thermal binder fiber, and the bi-component thermal binder fiber comprises polypropylene and polyethylene.
7. The absorbent article wherein the nonwoven material comprises a range of about 2% to about 5% by weight of the low contact, bi-component thermal binder fiber.
8. The absorbent article wherein the nonwoven material comprises an absorbent basis weight in a range of about 125 g/m² to about 250 g/m².
9. The absorbent article wherein the pad comprises a barrier film comprising polyvinyl alcohol (PVOH).
10. The absorbent article wherein the nonwoven material comprises up to 100% hemp fiber.
11. The absorbent article wherein the nonwoven material comprises greater than about 50% hemp fiber.
12. The absorbent article wherein the nonwoven material comprises a blend of materials comprising at least one of viscose rayon, cotton or polylactic acid (PLA) fibers.
13. An absorbent article that is compostable, comprising:
   a pad for feminine hygiene, the pad comprising a nonwoven material that is carded and comprises hemp and a hemp fiber length is in a range of about 40 mm to about 55 mm, and the nonwoven material comprises hydroentangling bonding.
14. The absorbent article wherein the pad comprises a barrier film comprising polylactic acid (PLA).
15. The absorbent article wherein the nonwoven material comprises up to 100% hemp fiber.
16. The absorbent article wherein the nonwoven material comprises greater than about 50% hemp fiber.
17. The absorbent article wherein the nonwoven material comprises a blend of materials comprising at least one of viscose rayon, cotton or polylactic acid (PLA) fibers.
18. The absorbent article wherein the nonwoven material is produced by an air forming process.
19. The absorbent article wherein the nonwoven material is hydrogen bonded.
20. The absorbent article wherein the nonwoven material comprises a bi-component thermal binder fiber.
21. The absorbent article wherein the nonwoven material comprises no greater than about 5% by weight of the bi-component thermal binder fiber, and the bi-component thermal binder fiber comprises polypropylene and polyethylene.
22. The absorbent article wherein the nonwoven material comprises a range of about 2% to about 5% by weight of the low contact, bi-component thermal binder fiber.
23. The absorbent article wherein the nonwoven material comprises an absorbent basis weight in a range of about 125 g/m² to about 250 g/m².
24. The absorbent article of claim 13, wherein the pad comprises a barrier film comprising polyvinyl alcohol (PVOH).
25. The absorbent article wherein the nonwoven material comprises a blend of materials comprising at least one of viscose rayon, cotton or polylactic acid (PLA) fibers.

This written description uses examples to disclose the embodiments, including the best mode, and also to enable those of ordinary skill in the art to make and use the invention. The patentable scope is defined by the claims, and can include other examples that occur to those skilled in the art. Such other examples are intended to be within the scope of the claims if they have structural elements that do not differ from the literal language of the claims, or if they include equivalent structural elements with insubstantial differences from the literal languages of the claims.

In the foregoing specification, the concepts have been described with reference to specific embodiments. However, one of ordinary skill in the art appreciates that various modifications and changes can be made without departing from the scope of the invention as set forth in the claims below. Accordingly, the specification and figures are to be regarded in an illustrative rather than a restrictive sense, and all such modifications are intended to be included within the scope of invention.

The description in the present application should not be read as implying that any particular element, step, or function is an essential or critical element that must be included in the claim scope. The scope of patented subject matter is defined only by the allowed claims. Moreover, none of the claims invokes 35 U.S.C. § 112(f) with respect to any of the appended claims or claim elements unless the exact words “means for” or “step for” are explicitly used in the particular claim, followed by a participle phrase identifying a function.

Benefits, other advantages, and solutions to problems have been described above with regard to specific embodiments. However, the benefits, advantages, solutions to problems, and any feature(s) that can cause any benefit, advantage, or solution to occur or become more pronounced are not to be construed as a critical, required, sacrosanct or an essential feature of any or all the claims.

After reading the specification, skilled artisans will appreciate that certain features which are, for clarity, described herein in the context of separate embodiments, can also be provided in combination in a single embodiment. Conversely, various features that are, for brevity, described in the context of a single embodiment, can also be provided separately or in any sub-combination.

Further, references to values stated in ranges include each and every value within that range.

What is claimed is:

1. An absorbent article that is flushable, comprising:
a tampon for feminine hygiene, the tampon comprising a nonwoven material comprising hemp, wherein the hemp has hemp fiber length in a range of about 8 mm to about 15 mm, and the nonwoven material comprises an absorbent ribbon and an overwrap for the tampon.

2. The absorbent article of claim 1, wherein the nonwoven material is produced by an air forming process.

3. The absorbent article of claim 1, wherein the nonwoven material is hydrogen bonded.

4. The absorbent article of claim 1, wherein the nonwoven material further comprises a water dispersible bi-component thermal binder fiber.

5. The absorbent article of claim 4, wherein the nonwoven material comprises about 2% to about 5% by weight of the water dispersible bi-component thermal binder fiber, and the bi-component thermal binder fiber comprises polypropylene and polyethylene.

6. The absorbent article of claim 1, wherein the hemp fiber length is 12 mm or less.

7. The absorbent article of claim 1, wherein the overwrap comprises a basis weight in a range of about 15 g/m² to about 25 g/m².

8. The absorbent article of claim 1, wherein the absorbent ribbon comprises a basis weight in a range of about 8 grams per meter of length to about 11 grams per meter of length.

9. The absorbent article of claim 1, wherein the absorbent ribbon comprises a basis weight of about 9.5 grams per meter of length.

10. The absorbent article of claim 1, wherein the overwrap comprises a carded or spunlaced nonwoven material.

11. The absorbent article of claim 1, wherein the absorbent ribbon comprises carded hemp, bleached cotton, viscose rayon, or a combination thereof.

12. The absorbent article of claim 11, wherein the absorbent ribbon comprises about 50% hemp and about 50% viscose rayon.

13. An absorbent article that is compostable, comprising:
a tampon for feminine hygiene, the tampon comprising a nonwoven material that is carded and comprises hemp with a hemp fiber length in a range of about 40 mm to about 55 mm, and wherein the nonwoven material is formed by hydroentangling bonding, and comprises an absorbent ribbon and an overwrap for the tampon.

14. The absorbent article of claim 13, wherein the nonwoven material comprises greater than about 50% hemp fiber.

15. The absorbent article of claim 13, wherein the nonwoven material comprises a blend of materials comprising at least one of viscose rayon, cotton, polylactic acid (PLA) fibers, or a combination thereof.

16. The absorbent article of claim 13, wherein the absorbent ribbon comprises a sliver ribbon having a width in a range of about 45 mm to about 55 mm.

17. An absorbent article that is flushable, comprising:
a pad for feminine hygiene, the pad comprising a nonwoven material comprising hemp and having a hemp fiber length in a range of about 8 mm to about 15 mm.

18. The absorbent article of claim 17, wherein the nonwoven material comprises greater than about 50% hemp fiber.

19. The absorbent article of claim 17, wherein the nonwoven material comprises an absorbent basis weight in a range of about 125 g/m² to about 250 g/m².

20. The absorbent article of claim 17, wherein the nonwoven material comprises about 2% to about 5% by weight of the water dispersible bi-component thermal binder fiber, and the bi-component thermal binder fiber comprises polypropylene and polyethylene.
